(19) United States
(12) Patent Application Publication
(10) Pub. No.: US 2012/0088731 A1
(43) Pub. Date: Apr. 12, 2012

(54) METHOD FOR TREATING PARKINSON'S DISEASE THROUGH REGULATING VDAC1 PROTEIN

(75) Inventors: Jongkyeong Chung, Daejeon (KR); Yongsung Kim, Seoul (KR)

(73) Assignee: SNU R&DB FOUNDATION, Seoul (KR)

(21) Appl. No.: 13/001,658

(22) PCT Filed: Oct. 14, 2010
(86) PCT No.: PCT/KR10/07052

§ 371 Date: Dec. 28, 2010

(30) Foreign Application Priority Data
Oct. 6, 2010 (KR) 10-2010-0097258

(51) Int. Cl. A61K 38/13; A61P 25/16; A61K 31/295; A61K 31/19

(52) U.S. Cl. 514/17.7; 514/557; 514/492

(57) ABSTRACT

The present invention provides a method for curing Parkinson's disease through regulating VDAC1 (voltage-dependent anion channel 1) protein. Specifically, the present invention provides a pharmaceutical composition for curing PD comprising a VDAC1 protein regulator. The VDAC1 protein regulators comprise an inhibitor of VDAC1 mRNA production, an inhibitor of mitochondrial permeability transition pore (mPTP) formation, and a mitochondrial calcium uptake inhibitor. In addition, the present invention provides a method for curing Parkinson's disease comprising administering a VDAC1 protein regulator to a subject with Parkinson's disease. The present invention could provide a method for curing Parkinson's disease through preventing and/or restoring mitochondrial dysfunction.

### FIELD OF THE INVENTION

The present invention relates to a method for curing Parkinson's disease, and more particularly to the method for curing Parkinson's disease through VDAC1 (voltage-dependent anion channel 1) protein.

### BACKGROUND OF THE INVENTION

Parkinson's disease (PD) is the second most prevalent neurodegenerative disease. There are over 4 million patients in the world and the incidence rate is rapidly increasing with rising elderly population. The symptoms of PD typically show progressive locomotive defects such as rigidity, tremor, bradykinesia of the limbs, and postural instability, and non-locomotive defects such as cognitive dysfunction, depression, sleep disorder, and pain.

In the aspect of anatomy, selective degeneration of dopaminergic (DA) neurons in the substantia nigra is the pathological hallmark of the disease. When 60-80% of dopamine level produced by DA neurons in the substantia nigra is deceased, extrapyramidal tracts cannot efficiently work and thus the symptoms of PD occur. Curable therapies have yet to be established because the exact cause of PD remains unknown, and currently there are only symptomatic therapies. As currently using or developing therapeutics, there are dopamine receptor agonists and dopamine precursors such as levodopa. In addition, COMT inhibitors and MAO-B inhibitors, which inhibit the metabolism of dopamine and thus maintain the concentration of dopamine in the brain, are used.

It is well known that the incidence of PD is associated with genetic and environmental factors as well as the aging. The genetic factors include alpha-synuclein, parkin, PINK1, UCH-L1, and DJ-1. Recently many studies on the mutations of PTEN-induced protein kinase 1 (PINK1) and parkin, the major causes of AR-JP (Autosomal Recessive Juvenile Parkinsonism), have given valuable insights on the pathogenic mechanisms of PD.

It is known that over 50% of AR-JP is associated with the mutation of parkin. Parkin protein has an ubiquitin homology domain in the N-terminus and two RING finger domains in the C-terminus. Like many other proteins having RING finger domains, Parkin is an E3 ligase that conjugates ubiquitin to target substrates, and it has been presumed that the mutation of parkin induces the abnormal accumulation of target substrates in the nigra and the striatum of patients. The studies have been actively done to find the substrate of Parkin E3 ligase and thus about 10 substrates such as alpha-synuclein, Parkin-associated endothelin receptor-like receptor (Pael-R), and CDCrel-1 have been proposed through these in vitro experiments.

In 2004, it was revealed that PINK1 is associated with PD [Valente et al. Hereditary early-onset Parkinson's disease caused by mutations in PINK1 (2004). Science 304, 1158-1160]. PINK1 protein has a mitochondrial targeting motif in the N-terminus and it is localized to the cytoplasm and mitochondria. In addition, PINK 1 protein has a serine/threonine kinase domain and shows autophosphorylation activity corresponding to the physiological activity of PINK1. PINK1 mutants found in PD patients show decreased autophosphorylation activity.

In 2006, the present inventors revealed that PINK1 plays an essential role in the protection of mitochondrial integrity, and that PINK1 and Parkin make a single signaling pathway with Parkin acting downstream of PINK1 [Park et al. Mitochondrial dysfunction in Drosophila PINK1 mutants is complemented by parkin (2006). Nature 441, 1157-1161]. In addition, it was revealed that Parkin is deeply related to the regulation of the mitochondria and there is high possibility that the target substrate is a mitochondrial protein. These results are on the contrary of the previously known substrates of Parkin, as they localize in the cytoplasm. However, it would be worth to find the new substrates of Parkin among mitochondrial proteins because the present inventors obtained the results mainly from in vivo studies and thus the credibility to the results is very high.

Based on these new findings, the inventors have tried to discover a novel substrate of Parkin in mitochondria, and also have identified regulators for the substrate which have a potential to cure PD.

### SUMMARY OF THE INVENTION

The object of the present invention is to provide a method for curing PD through preventing and/or restoring mitochondrial dysfunction. In addition, the present invention is to provide not a symptomatic therapy but a curable therapy through regulating VDAC1 protein in mitochondria.

In one embodiment of the present invention, there is provided a pharmaceutical composition for curing PD comprising a VDAC1 protein regulator. The VDAC1 protein regulator comprises an inhibitor of VDAC1 mRNA production such as asiatic acid, an inhibitor of mitochondrial permeability transition pore (mPTP) formation such as cyclosporine A, and a mitochondrial calcium uptake inhibitor such as Ru360.

In another embodiment of the present invention, there is provided a method for curing PD comprising administering a VDAC1 protein regulator to a subject with PD. The VDAC1 protein regulator is the same as mentioned above.

### BRIEF DESCRIPTION OF THE DRAWINGS

FIG. 1 shows that VDAC1 is a target substrate of Parkin.

FIG. 2 shows that VDAC1 protein level is increased when SK-N-BE(2)C cells were transfected with parkin siRNA.

FIG. 3 shows the mitochondrial morphology at 72 hours after SK-N-BE(2)C cells were transfected with VDAC1 siRNA.

FIG. 4 shows the effect of VDAC1 protein regulators on mitochondrial fragmentation in neurons transfected with parkin shRNA.

FIG. 5 shows the quantification of FIG. 4.

FIG. 6 shows VDAC1 protein level at 72 hours after SK-N-BE(2)C cells were transfected with PINK siRNA.

FIG. 7 shows the effect of VDAC1 protein regulators on mitochondrial fragmentation in neurons transfected with PINK shRNA.

FIG. 8 shows the quantification of FIG. 7.

FIG. 9 shows the effect of VDAC1 protein regulators on the mitochondrial defects of parkin null mouse mesencephalic dopaminergic (mDA) neurons.

FIG. 10 shows the quantification of FIG. 9.

FIG. 11 shows the effect of VDAC1 protein regulators on the synaptic activity of parkin null mDA neurons.

FIG. 12 shows the quantification of FIG. 11 (FIG. 12a: frequency of mEPSC, FIG. 12b: amplitude of mEPSC).

### DETAILED DESCRIPTION OF THE INVENTION

The present invention provides a method for curing PD due to mitochondrial dysfunction through regulating VDAC1 protein. Specifically, the present invention provides a pharmaceutical composition for curing PD comprising VDAC1 protein regulators. The VDAC1 protein regulator; comprise an inhibitor of VDAC1 mRNA production, an inhibitor of mitochondrial permeability transition pore (mPTP) formation, and a mitochondrial calcium uptake inhibitor.

Furthermore, the present invention provides a method for curing PD comprising administering VDAC1 protein regulators to a subject with PD. The subject in the present invention comprises human and non-human mammals such as mouse, rat, dog, cat, horse, and cow.

It has been revealed that the pathological mechanism of PD is associated with mitochondrial dysfunction in Drosophila model research. In addition, it has been known that PINK1 phosphorylates Parkin and the phosphorylated Parkin is translocated to mitochondria.

Parkin normally locates in the cytoplasm. However, Parkin translocates to mitochondria when the mitochondrial dysfunction is induced after the uncoupler carbonyl cyanide m-chlorophenylhydrazone (CCCP) treatment, finally, removes the dysfunctional mitochondria from cells. PINK1 is required in the translocation of Parkin. Parkin induces polyubiquitination and degradation of a target substrate.

The present inventors presumed that Parkin would ubiquitinate and lead an important mitochondrial protein to the degradation. Based on this hypothesis, the present inventors performed to clone several mitochondrial proteins and screened them to find a substrate of Parkin. Specifically, the inventors performed polyubiquitination reaction on several mitochondrial proteins and found that VDAC1, a mitochondrial protein, is a target substrate of Parkin. In addition, it was found that mutant Parkin in PD patients cannot induce VDAC1 ubiquitination and degradation. Furthermore, it was revealed that VDAC1 protein regulators can prevent and/or restore mitochondrial dysfunction.

BE(2)C, a human neuroblastoma cell line, secretes dopamine and frequently used in PD research. The present inventor demonstrated that VDAC1 protein level was increased at 72 hours after BE(2)C was transfected with Parkin siRNA (short interfering RNA).

Taken together, VDAC1 is a mitochondrial substrate of Parkin and Parkin induces the degradation of VDAC1. VDAC1, which is located in the outer membrane of mitochondria, plays an important role in energy production and mitochondrial homeostasis maintenance. Specifically, VDAC1 is involved in delivering Ca²⁺, pyruvate, and ADP to the mitochondria and releasing the produced ATP. In addition, VDAC1 is a major component of mitochondrial permeability transition pore (mPTP). The opening of mPTP promotes mitochondrial dysfunction and fragmentation, finally induces apoptosis. Recently it has been revealed that overexpression of VDAC1 causes cell death. As described above, VDAC1 plays an essential role in mitochondrial homeostasis maintenance and also it can induce cell death. When cell death occurs, mitochondria morphology is changed: the elongated mitochondria are fragmented, and the openings of mPTP and membrane potential loss are induced, leading to dysfunctional mitochondria.

Considering these, it would be presumed that the deficiency of Parkin increases VDAC1 protein level and thus mitochondria morphology is changed. Regarding this, the present inventors demonstrated that mitochondria in neurons transfected with parkin shRNA (short hairpin RNA) are significantly fragmented compared to the control. Furthermore, it was found that an inhibitor of VDAC1 mRNA production such as asiatic acid (AA), an inhibitor of mitochondrial permeability transition pore (mPTP) formation such as cyclosporine A (CsA), and a mitochondrial calcium uptake inhibitor such as Ru360 prevent mitochondrial fragmentation induced by parkin shRNA.

To confirm this result, parkin null mouse mDA neurons were treated with AA, CsA, or Ru360. As a result, the mitochondrial fragmentation in parkin null mDA neurons was restored up to the level of heterozygotic neurons. In addition, it was remarkable that the treatment of AA, CsA, and Ru360 to parkin null mDA neurons restored the synaptic activity up to the level of heterozygotic neurons.

Furthermore, it was demonstrated that PINK1, an upstream regulator of Parkin, induced VDAC1 degradation, and the treatment of AA, CsA, and Ru360 to PINK1-downregulated neurons restored the mitochondrial fragmentation up to the normal level.

Taken together, it is expected that VDAC1 protein regulators such as AA and its analogues, Ru360 and its analogues, and CsA and its analogues can be used to develop therapeutics for curing VDAC1 related diseases such as Alzheimer's disease and Lou Gehrig's disease as well as PD.

### EXAMPLES

In the following, the present invention is described in detail through experiments. The experiments are not intended to limit the technical spirit of the present invention, but are intended to describe the invention.

### Experiment 1

### Mitochondrial Substrate of Parkin: VDAC1

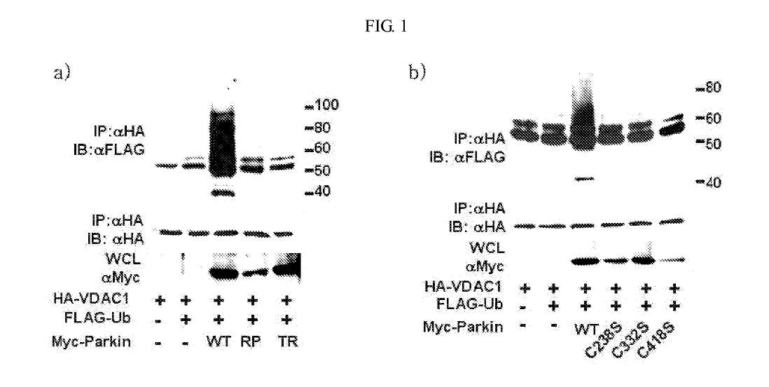

HEK293T cells were transiently transfected with pcDNA3.1 HA-VDAC1 (provided by Dr. Ulf R. Rapp, Institut fuer Medizinische Strahlenkunde and Zellforschung), pcDNA3 FLAG-ubiquitin, and pcDNA3.1 Myc-Parkin cDNA (Invitrogen) by using Lipofectamine Plus Reagent (Invitrogen). Following 48 hours of transfection, the cells were lysed and the immunoprecipitation was performed by using HA-antibody (12CA5 HA, Roche) and Protein G (Amersham). Immunoblot analyses were completed using anti-FLAG and -HA antibodies. The results are shown in FIG. 1 (IP: immunoprecipitation, IB: immunoblot, WCL: whole cell lysates, upper panel: ubiquitination level of VDAC1 protein, middle panel: expression level of VDAC1 protein, lower panel: expression level of Parkin protein).

As shown in FIG. 1, both of the well-known mutant forms of Parkin, RP and TR, did not induce the ubiquitination and degradation of VDAC1, and while wild type Parkin (WT) did. In addition, the structural mutants of Parkin (C238S, C332S, C418S) did not induce the degradation of VDAC1 (FIG. 1b).

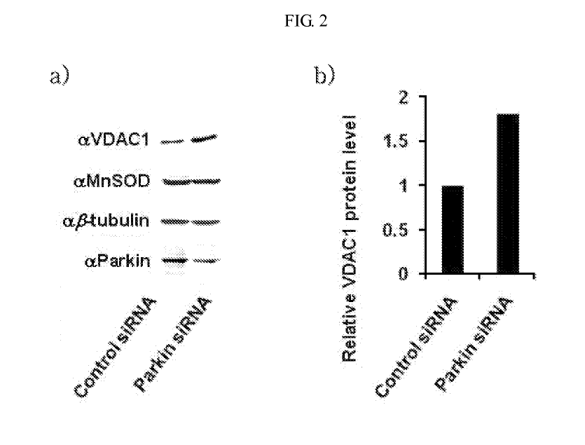

Furthermore, SK-N-BE(2)C cells were transfected with control siRNA (D-001210-01-20, Dharmacon) or parkin siRNA (M-003603-00, Dharmacon) by using XtremeGene (Roche). After 72 hours of transfection, cells were lysed for immunoblot analyses. The results are shown in FIG. 2. As shown in FIG. 2, transfection with parkin siRNA increased the amount of endogenous VDAC1 protein level (FIG. 2a) and the relative VDAC1 protein level was almost doubled (FIG. 2b).

### Experiment 2

### The Effect of VDAC1 on Mitochondrial Morphology

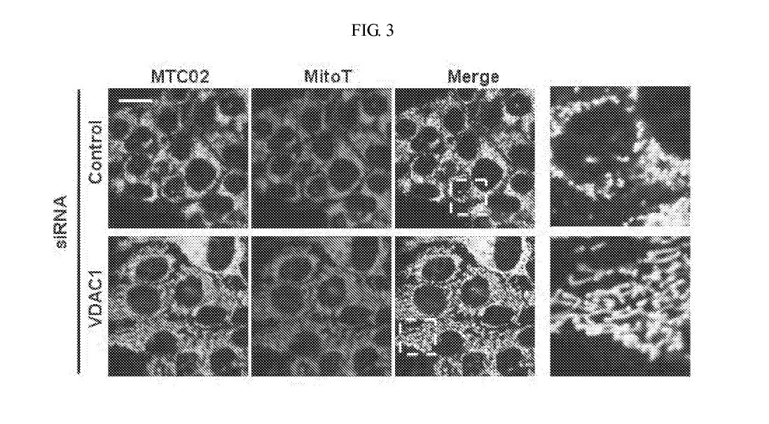

SK-N-BE(2)C cells were transfected with control siRNA or VDAC1 siRNA (M-019764-00, Dharmacon) by using XtremeGene (Roche). After 72 hours of transfection, cells were treated with Mitotracker (Invitrogen) and fixed with 2% paraformaldehyde. In addition, cells were stained with anti-MTC02 (mitochondria staining, green color) and observed by confocal microscopy (Carl Zeiss, Product No. LSM510). The results are shown in FIG. 3.

As shown in FIG. 3, VDAC1 knockdown by siRNA induced mitochondrial fusion. This result indirectly proves that Parkin acting downstream of PINK induces mitochondrial fusion by the degradation of VDAC1.

### Experiment 3

### The Effect of VDAC1 Protein Regulators on the Neurons Transfected with Parkin shRNA

Cultured rat hippocampal neurons were transfected with parkin shRNA [pSUPER neo/gfp vector (Oligoengine), shRNA sequence (Dharmacon)] at 13 days in vitro (DIV), and VDAC1 protein regulators were treated [AA: 48 hours, CsA: 24 hours, Ru360: 24 hours, VDAC1 shRNA: 72 hours transfection (pSUPER neo/gfp vector, the shRNA sequence from Abu-Hamid et al. Proc. Natl. Acad. Sci. USA. 103, 5787-5792)].

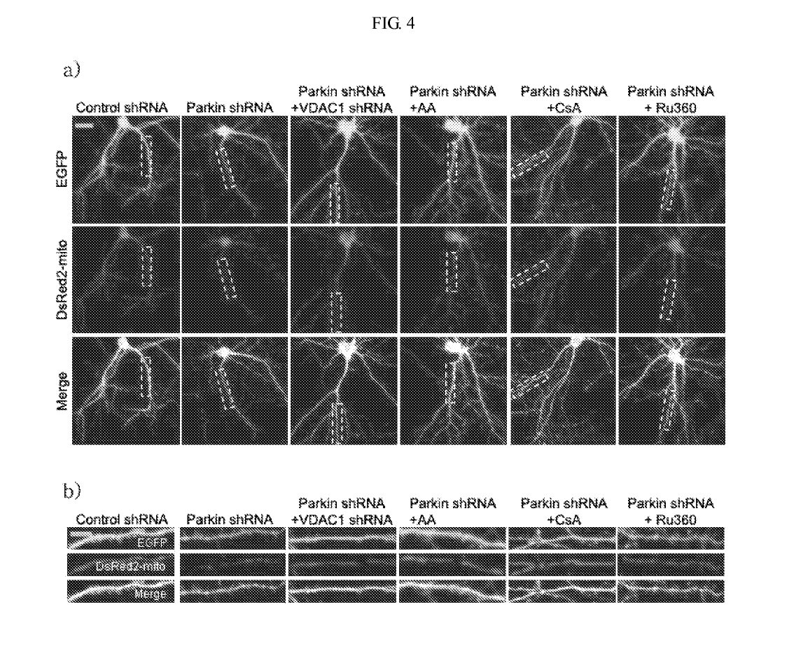

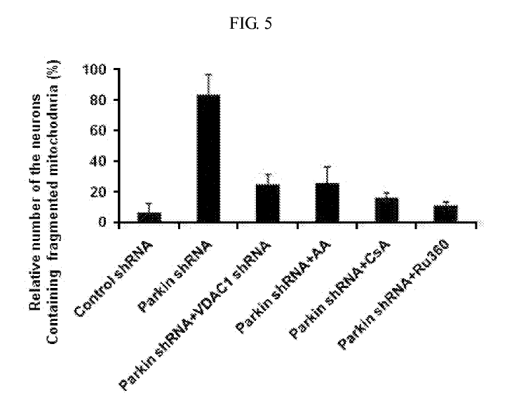

After 72 hours of transfection, immunocytochemistry was performed and the results are shown in FIG. 4 and FIG. 5. FIG. 4b shows magnified images of the boxed regions in FIG. 4a and FIG. 5 is the quantification of FIG. 4a. EGFP was used as a fluorescence marker to confirm the transfection of neuron with shRNA vector, and DsRed2-mito [pDsRed2-mito (Clontech)] was used as a fluorescence marker to observe mitochondria in neurons.

As shown in FIG. 4, the mitochondrial fragmentation phenotype was specifically mediated by parkin shRNA. Furthermore, AA, CsA, Ru360, and shRNA expression of VDAC1 suppressed the dendritic mitochondrial fragmentation induced by parkin knockdown. In addition, the relative numbers of the neurons containing fragmented mitochondria in FIG. 5 clearly demonstrated that reducing VDAC1 dosage by VDAC1 shRNA and AA, or inhibiting its functions by CsA and Ru360 prevents dendritic mitochondrial fragmentation in parkin-knockdowned hippocampal neurons and/or restores the fragmented mitochondria to normal elongated morphology.

### Experiment 4

### The Effect of PINK1 Knockdown on VDAC1

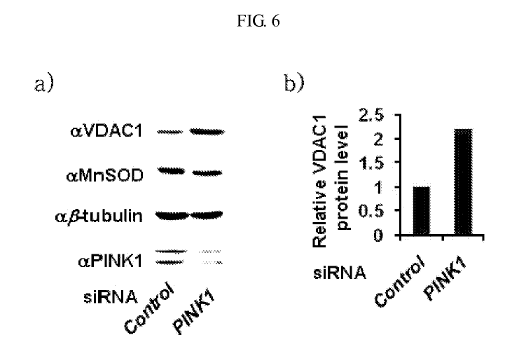

SK-N-BE(2)C cells were transfected with control siRNA or PINK1 siRNA (M-004030-02, Dharmacon) by using XtremeGene (Roche). After 72 hours of transfection, the cells were lysed [Lysis buffer: lysis buffer A (20 mM Tris pH 7.5, 100 mM NaCl, 1 mM EDTA, 2 mM EGTA, 50 mM β-glycerophosphate, 50 mM NaF, 1 mM sodium vanadate, 2 mM DTT, 1 mM PMSF, 10 g/ml leupeptin, 1 g/ml pepstatin A, 1% Triton X-100)] and immunoblot analyses were performed (Bradford assay was used for protein quantification). The results are shown in FIG. 6.

As shown in FIG. 6, PINK1 knockdown by siRNA increased the endogenous VDAC1 protein level (FIG. 6a) and the relative VDAC1 protein level was increased over twice by PINK1 knockdown (FIG. 6b).

### Experiment 5

### The Effect of VDAC1 Protein Regulators on the Neurons Transfected with PINK1 shRNA

Cultured rat hippocampal neurons were transfected with PINK1 shRNA [pSUPER neo/gfp vector (Oligoengine), shRNA sequence (Dharmacon)] at 13 days in vitro (DIV), and VDAC1 protein regulators were treated (AA: 48 hours, CsA: 24 hours, Ru360: 24 hours, VDAC1 shRNA: 72 hours transfection).

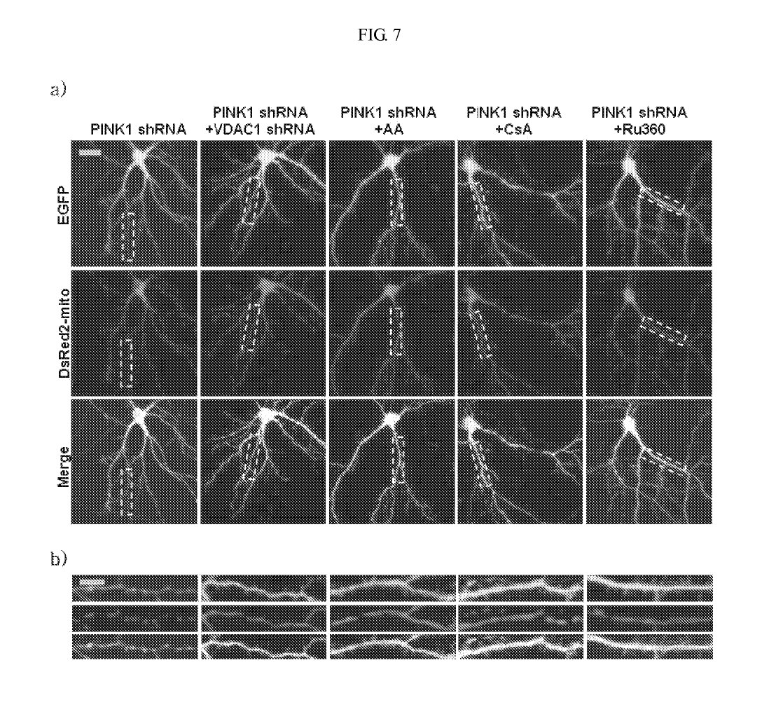

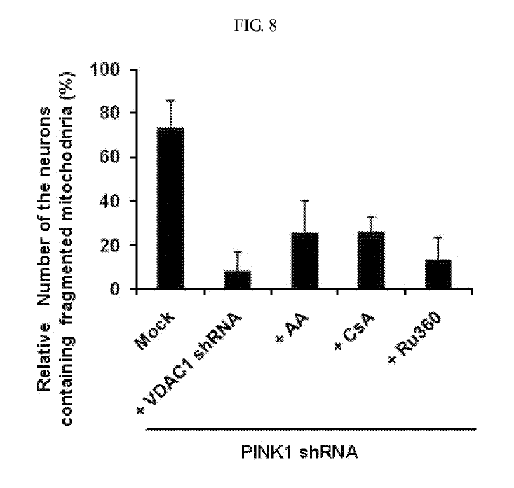

After 72 hours of transfection, immunocytochemistry was performed and the results are shown in FIG. 7 and FIG. 8. FIG. 7b shows magnified images of the boxed regions in FIG. 7a and FIG. 8 is the quantification of FIG. 7a.

As shown in FIG. 7, the mitochondrial fragmentation phenotype is specifically mediated by PINK1 shRNA. Furthermore, AA, CsA, Ru360, and shRNA expression of VDAC1 suppressed the dendritic mitochondrial fragmentation induced by knockdown of PINK1. In addition, the relative number of the neurons containing fragmented mitochondria in FIG. 8 clearly demonstrates that reducing VDAC1 dosage by VDAC1 shRNA and AA, or inhibiting its functions by CsA and Ru360 prevents dendritic mitochondrial fragmentation in PINK1-knockdowned hippocampal neurons and/or restores the fragmented mitochondria to normal elongated morphology.

### Experiment 6

### The Effect of VDAC1 Protein Regulators on the Mitochondrial Defects of Parkin Null Mouse mDA Neurons

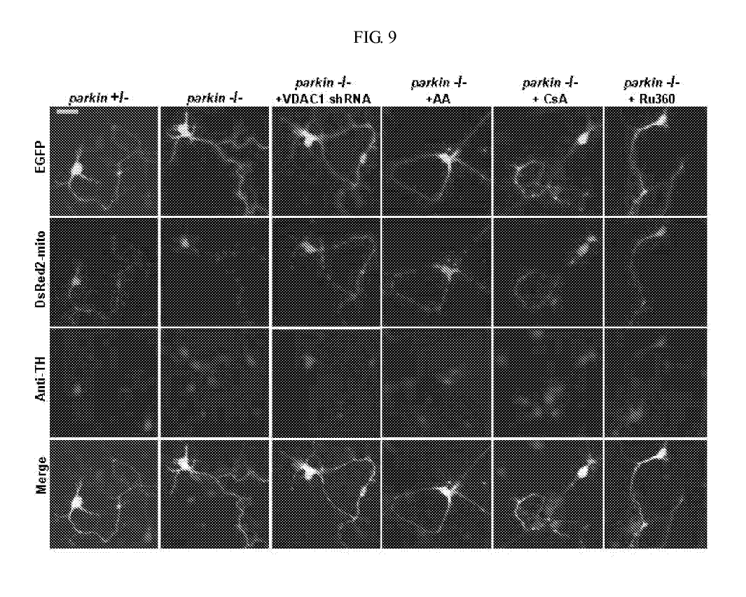

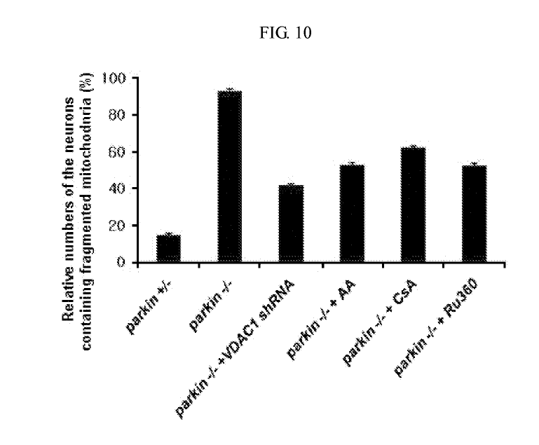

Primary mouse DA neuron cultures were prepared from embryonic day (E) 13.5 mouse midbrain of parkin heterogeneous(+/−) and null(−/−) embryos [Neurobasal Media (Invitrogen)]. At days 7 in vitro (DIV), immunocytochemistry was performed and the results are shown in FIG. 9. As shown in FIG. 9, the mDA neurons from parkin null mice (Parkin −/−) have fragmented mitochondria. Intriguingly, the mDA neurons treated with AA, CsA, or RU360 for 24 hours showed a significantly restored level of the elongated mitochondria. FIG. 10 shows the relative numbers of the neurons containing fragmented mitochondria (the quantification of FIG. 9). FIG. 10 clearly demonstrates that the mDA neurons from parkin null mice have fragmented mitochondria compared to the neurons from heterozygous littermates. In addition, reducing VDAC1 dosage by AA or inhibiting its functions by CsA and Ru360 significantly prevented mitochondrial fragmentation in parkin null mouse mDA neurons.

### Experiment 7

### The Effect of VDAC1 Protein Regulators on the Synaptic Activity of Parkin Null Mouse mDA Neurons

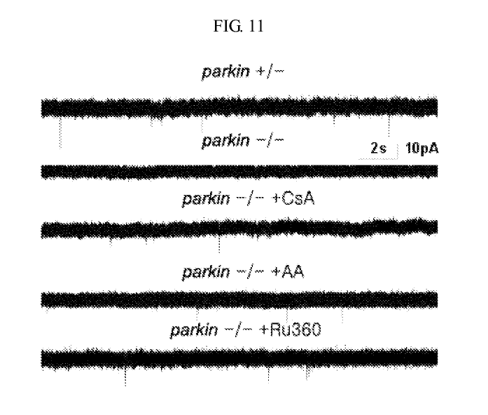

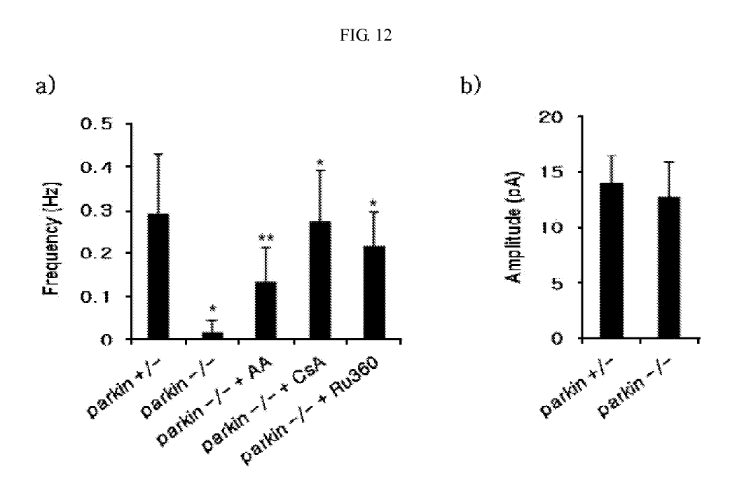

It was examined whether PINK1-Parkin-VDAC1 pathway regulates the synaptic activity of the mDA neurons. To this end, the frequency and amplitude of miniature excitatory postsynaptic currents (mEPSCs) of parkin heterozygous and null mouse mDA neurons were measured. The experimental condition was the same as Experiment 6 and the results are shown in FIG. 11 and FIG. 12. FIG. 12 shows the quantification of the frequency (a) and the amplitude (b) from FIG. 11. As shown in FIG. 12, homozygotic loss of parkin in the mDA neurons resulted in a decrease in the frequency of mEPSCs, and, remarkably, the treatment of AA, CsA, and Ru360 to parkin null mDA neurons restored the frequency of mEPSCs up to the level of heterozygotic neurons.

It is understood to a person skilled in the art that the above description of the present invention is susceptible to various modifications, changes and adaptations, and the same are intended to be comprehended within the meaning and range of equivalents of the appended claims. Therefore, the embodiments and attached drawings disclosed in the present invention are not intended to limit the technical spirit of the present invention, but are intended to describe the invention. The technical spirit of the present invention is not limited to such embodiments and drawings.

1-7. (canceled)

8. A method for treating Parkinson's disease comprising administering an effective amount of a pharmaceutical composition comprising a VDAC1 (voltage-dependent anion channel 1) protein regulator to a subject with Parkinson's disease.

9. The method according to claim 8, wherein the VDAC1 protein regulator is an inhibitor of VDAC1 mRNA production, an inhibitor of mitochondrial permeability transition pore (mPTP) formation, or a mitochondrial calcium uptake inhibitor.

10. The method according to claim 9, wherein the inhibitor of VDAC1 mRNA production is asiatic acid.

11. The method according to claim 9, wherein the inhibitor of mPTP formation is cyclosporine A.

12. The method according to claim 9, wherein the mitochondrial calcium uptake inhibitor is Ru360.
